(19) United States
(12) Patent Application Publication
(10) Pub. No.: US 2024/0257954 A1
(43) Pub. Date: Aug. 1, 2024

(54) METHODS, SYSTEMS, AND COMPUTER PROGRAM PRODUCTS FOR DISCOVERING PATIENT BENEFITS UNDER A HEALTH CARE COVERAGE PLAN

(71) Applicant: Optum, Inc., Minnetonka, MN (US)

(72) Inventors: Arien Malec, Oakland, CA (US); Deas Richardson, IV, Mt. Pleasant, SC (US); Kirk Sudduth, Hendersonville, TN (US)

(21) Appl. No.: 18/162,157

(22) Filed: Jan. 31, 2023

(51) Int. Cl. G16H 40/20; G06Q 40/08

(52) U.S. Cl. CPC G16H 40/20; G06Q 40/08

(57) ABSTRACT

A method includes receiving a patient health care benefit query from a requestor; generating a health care coverage eligibility request for the patient; communicating the health care coverage eligibility request to a payor; electronically updating a benefit rule repository with information from an eligibility confirmation response from the payor; adjudicating one or more rules, which are based on a health care coverage policy provided by the payor for the patient, contained in the benefit rule repository; determining a benefit eligibility result for the patient based on adjudicating the one or more rules; and communicating the benefit eligibility result to the requestor.

### FIELD

The present inventive concepts relate generally to health care systems and services and, more particularly, to determining benefit eligibility for a patient covered by a health plan.

### BACKGROUND

A health care service provider may query a health care payment plan administrator, e.g., a private insurance entity, government insurance entity, and/or a medical expense sharing organization, which may be referred to as a “payor,” to determine a patient's eligibility under a payment plan or coverage plan offered by the payor. This eligibility query may be performed at various stages during the patient care process, such as, for example, in advance of a patient's appointment, when the patient arrives for the appointment, and/or when generating a bill after a patient has been cared for by a health care service provider. The payment plan or coverage plan eligibility determination is used to ensure that the patient is billed correctly and receives all the benefits that the patient is entitled to. Determining the particular benefits that a patient is entitled to under a particular plan, however, may be more difficult as these benefits may be described in the details of the coverage plan offered by the payor, which may not be readily accessible. Moreover, many benefits come with conditions, such as only being available if justified by a medical necessity or only being available if prior authorization by the payor is obtained. Analyzing the payor information may require experts that have accumulated knowledge and/or special training in interpreting provisions of a health care plan to understand the details associated with benefit eligibility.

### SUMMARY

According to some embodiments of the inventive concept, a method comprises: receiving a patient health care benefit query from a requestor; generating a health care coverage eligibility request for the patient; communicating the health care coverage eligibility request to a payor; electronically updating a benefit rule repository with information from an eligibility confirmation response from the payor; adjudicating one or more rules, which are based on a health care coverage policy provided by the payor for the patient, contained in the benefit rule repository; determining a benefit eligibility result for the patient based on adjudicating the one or more rules; and communicating the benefit eligibility result to the requestor.

In other embodiments, the method further comprises: processing a benefit information source associated with the payor using Natural Language Processing (NLP) to extract rules therefrom; and updating the benefit rule repository with the rules extracted from the benefit information source.

In still other embodiments, the benefit information source comprises a website including payor benefit information, an electronic policy document including payor benefit information, and/or an electronic business procedures document including payor benefit information.

In still other embodiments, the method further comprises: manually processing a benefit information source associated with the payor to extract rules therefrom; and updating the benefit rule repository with the rules extracted from the benefit information source.

In still other embodiments, the benefit information source comprises a website or paper document including payor benefit information, an electronic or paper policy document including payor benefit information, and/or an electronic or paper business procedures document including payor benefit information.

In still other embodiments, the method further comprises: updating the benefit rule repository with rules based on historical eligibility confirmation responses from the payor for historical patients; and updating the benefit rule repository with rules based on historical prior-authorization for care responses from the payor for historical patients.

In still other embodiments, the operations of receiving the patient health care benefit query, generating the health care coverage eligibility request, communicating the health care coverage eligibility request, electronically updating the benefit rule repository, adjudicating the one or more rules, determining the benefit eligibility result, and communicating the benefit eligibility result are performed in real-time.

In still other embodiments, the benefit eligibility result comprises one or more conditions associated therewith.

In still other embodiments, the one or more conditions are based on clinical information associated with the patient, whether the benefit qualifies as a medical necessity, and/or whether the payor requires pre-authorization for the benefit.

In still other embodiments, generating the health care coverage eligibility request for the patient comprises: processing a benefit information source associated with the payor using Natural Language Processing (NLP) to extract rules therefrom or manually processing the benefit information source associated with the payor to extract the rules therefrom; and updating an eligibility request rule repository with the rules extracted from the benefit information source.

In still other embodiments, generating the health care coverage eligibility request for the patient further comprises: updating the eligibility request rule repository with rules based on historical responses from the payor for historical patients; wherein the historical responses from the payor comprise historical eligibility confirmation responses from the payor for historical patients, historical prior-authorization for care responses from the payor for historical patients, eligibility query error responses from the payor for historical patients, and eligibility default responses from the payor for historical patients.

In still other embodiments, generating the health care coverage eligibility request for the patient further comprises: evaluating the health care benefit query based on the eligibility request rule repository to generate an evaluation of the health care benefit query; communicating a recommendation for revising the health care benefit query based on the evaluation of the health care benefit query; and receiving a revised health care benefit query from the requestor; wherein communicating the health care coverage eligibility request to the payor comprises communicating the health care coverage eligibility request to the payor based on the revised health care benefit query from the requestor.

In some embodiments of the inventive concept, a system comprises: a processor; and a memory coupled to the processor and comprising computer readable program code embodied in the memory that is executable by the processor to perform operations comprising: receiving a patient health care benefit query from a requestor; generating a health care coverage eligibility request for the patient; communicating the health care coverage eligibility request to a payor; electronically updating a benefit rule repository with information from an eligibility confirmation response from the payor; adjudicating one or more rules, which are based on a health care coverage policy provided by the payor for the patient, contained in the benefit rule repository; determining a benefit eligibility result for the patient based on adjudicating the one or more rules; and communicating the benefit eligibility result to the requestor.

In further embodiments, the operations further comprise: processing a benefit information source associated with the payor using Natural Language Processing (NLP) to extract rules therefrom; and updating the benefit rule repository with the rules extracted from the benefit information source.

In still further embodiments, the benefit information source comprises a website including payor benefit information, an electronic policy document including payor benefit information, and/or an electronic business procedures document including payor benefit information.

In still further embodiments, the operations further comprise: manually processing a benefit information source associated with the payor to extract rules therefrom; and updating the benefit rule repository with the rules extracted from the benefit information source.

In still further embodiments, the benefit information source comprises a website or paper document including payor benefit information, an electronic or paper policy document including payor benefit information, and/or an electronic or paper business procedures document including payor benefit information.

In still further embodiments, the operations further comprise: updating the benefit rule repository with rules based on historical eligibility confirmation responses from the payor for historical patients; and updating the benefit rule repository with rules based on historical prior-authorization for care responses from the payor for historical patients.

In some embodiments of the inventive concept, a computer program product comprises: a non-transitory computer readable storage medium comprising computer readable program code embodied in the medium that is executable by a processor to perform operations comprising: receiving a patient health care benefit query from a requestor; generating a health care coverage eligibility request for the patient; communicating the health care coverage eligibility request to a payor; electronically updating a benefit rule repository with information from an eligibility confirmation response from the payor; adjudicating one or more rules, which are based on a health care coverage policy provided by the payor for the patient. contained in the benefit rule repository; determining a benefit eligibility result for the patient based on adjudicating the one or more rules; and communicating the benefit eligibility result to the requestor.

In other embodiments, the operations further comprise: processing a benefit information source associated with the payor using Natural Language Processing (NLP) to extract rules therefrom; and updating the benefit rule repository with the rules extracted from the benefit information source.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter and be protected by the accompanying claims.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram that illustrates a communication network including a benefit discovery system in accordance with some embodiments of the inventive concept;

FIG. 2A is a block diagram that illustrates the benefit discovery system in accordance with some embodiments of the inventive concept;

FIG. 2B is a block diagram that illustrates a benefit query recommendation system in accordance with some embodiments of the inventive concept;

FIGS. 3 and 4 are flowcharts that illustrate operations for discovering benefits under a health care coverage plan in accordance with further embodiments of the inventive concept;

FIG. 5 is a flowchart that illustrates operations for recommending revisions to a benefit query in accordance with some embodiments of the inventive concept;

FIG. 6 is a data processing system that may be used to implement a benefit discovery system in accordance with some embodiments of the inventive concept; and

FIG. 7 is a block diagram that illustrates a software/hardware architecture for use in in a benefit discovery system in accordance with some embodiments of the inventive concept.

### DETAILED DESCRIPTION

In the following detailed description, numerous specific details are set forth to provide a thorough understanding of embodiments of the inventive concept. However, it will be understood by those skilled in the art that embodiments of the inventive concept may be practiced without these specific details. In some instances, well-known methods, procedures, components, and circuits have not been described in detail so as not to obscure the inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care products and/or services to a patient.

Embodiments of the inventive concept are described herein in the context of providing a patient benefits discovery system that processes patient benefits information from one or more sources to generate a benefits rule repository in which rules may be adjudicated using an underlying rule ontology or domain specific model. The representation of the domain specific model may be through a set of custom JavaScript Object Notation (JSON) model representations or in a standard, such as Resource Description Framework (RDF) or Web Ontology Language (OWL) in accordance with different embodiments of the inventive concept. The representation language may make inherent semantic information clear through the standard, but the model is specific to the patient coverage eligibility and benefits domain. The patient benefits discovery system described herein may be configured to transform a memory of a computer system to include one or more data structures, such as, but not limited to, arrays, extensible arrays, linked lists, binary trees, balanced trees, heaps, stacks, and/or queues. These data structures can be configured or modified through the rule generation and/or adjudication process to improve the efficiency of a computer system when the computer system operates in an inference mode to make an inference or prediction with respect to patient coverage eligibility and/or benefits entitlement in response to input information or data provided thereto.

Some embodiments of the inventive concept stem from a realization that while eligibility queries for determining whether a patient is covered under a particular payor's health plan are generally common in the delivery of health care services and products, determining whether a patient is eligible for particular benefits under the coverage plan may be more complicated. Whether a patient is entitled to a particular benefit may be based on information contained in coverage plan documents and may also have conditions associated therewith making it difficult to determine benefit eligibility. Some embodiments of the inventive concept may provide a benefit discovery system that may be configured to provide a coverage eligibility and benefits domain specific model based on rules that are generated or derived from information obtained from one or more sources including, but not limited to, payor documentation and coverage eligibility responses from a payor. The rules generated or updated based on the payor documentation may be a manual process and/or Natural Language Processing (NLP) may be used to facilitate processing electronic versions of payor documentation and information pertaining to coverage and benefits. Upon receipt of a benefit query from a requestor, the rules may be adjudicated to determine whether the patient may be entitled to a benefit including, in some embodiments, identifying any potential conditions that may impact eligibility for the benefit. By collecting unstructured information from multiple disparate sources, such as coverage eligibility responses and various payor documentation sources, and creating a structured, domain specific model based on rules that can be adjudicated, benefit eligibility queries can be processed in a much more efficient, consistent, and reliable way. Manually or electronically searching payor documentation or coverage eligibility confirmation responses from a payor for benefit information may be time consuming and resource intensive. Moreover, the ultimate determination of whether a patient is eligible for a benefit may be left to interpretation by a user, which can lead to inconsistent and/or inaccurate results.

Other embodiments of the inventive concept stem from a realization that the party that submits a benefits eligibility request may not generate the most accurate or useful query. Benefits eligibility inquiries to payors and responses thereto use the X12 specification, which is a specification for electronic data interchange relating to business transactions. The eligibility inquiry and response thereto are known as 270 requests and 271 responses under the X12 specification. The X12 specification is not a standard; however, which can result in payors and requestors interpreting the various messages and codes in different ways. This can result in requestors receiving error messages in response to what they believe is a valid inquiry, a payor misinterpreting an inquiry and returning a valid response for a different inquiry, but which is inapplicable to the inquiry submitted by the requestor, and/or a payor sending a default response, which may be interpreted by the requestor as a valid response, but which may be inapplicable to the inquiry submitted. Some embodiments of the inventive concept may provide a benefit query recommendation system that may be configured to provide a query recommendation model based on rules that are generated or derived from information obtained from one or more sources including, but not limited to, payor documentation and coverage eligibility responses from a payor. These historical responses from a payor may include, but are not limited to, eligibility confirmation responses, prior-authorization for care responses, error responses (e.g., the inquiry is not valid or unrecognizable), and/or default responses (e.g., the payor returns a non-error response to the requestor as a default, but the default response may be inapplicable to the inquiry made by the requestor). The health care benefit query from the requestor can be evaluated based on the rules generated for the particular payor and a recommendation can be returned to the requestor indicating how the query may be modified to obtain a more accurate and useful response from the payor. The benefit query recommendation system embodiments may work in conjunction with or independent of the benefit discovery system embodiments as described herein.

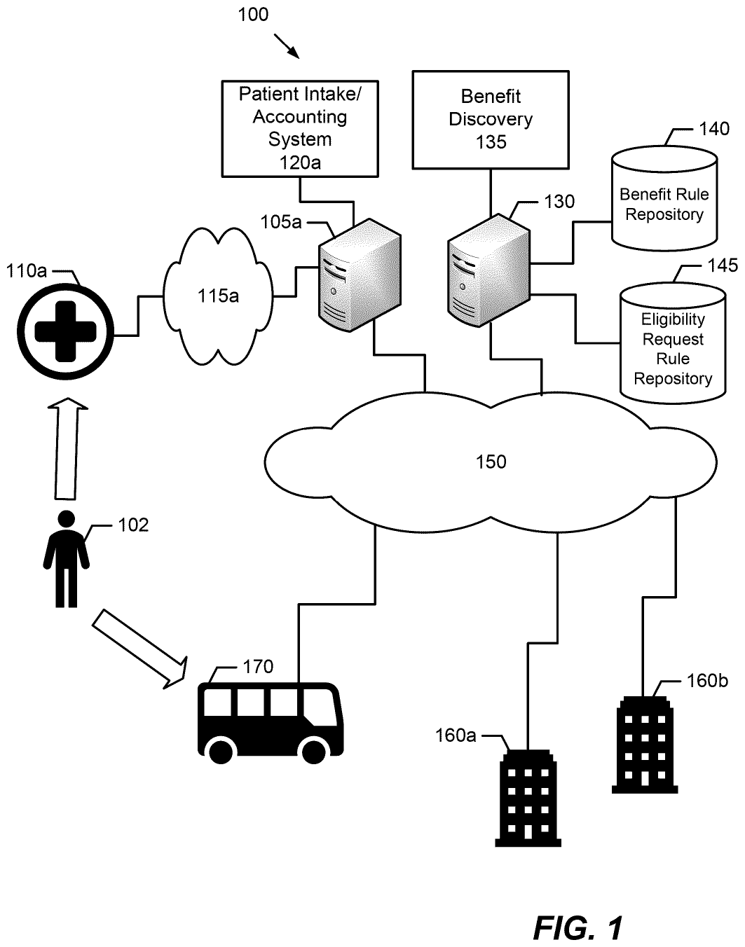

Referring to FIG. 1, a communication network 100 including a benefit discovery system, in accordance with some embodiments of the inventive concept, comprises one or more health care provider facilities or practices 110a. Each health care provider facility or practice may represent various types of organizations that are used to deliver health care services to patients via health care professionals, which are referred to generally herein as “providers.” The providers may include, but are not limited to, hospitals, medical practices, mobile patient care facilities, diagnostic centers, lab centers, pharmacies, and the like. The providers may operate by providing health care services for patients and then invoicing one or more payors 160a and 160b for the services rendered. The payors 160a and 160b may include, but are not limited to, providers of private insurance plans, providers of government insurance plans (e.g., Medicare, Medicaid, state, or federal public employee insurance plans), providers of hybrid insurance plans (e.g., Affordable Care Act plans), private of private medical cost sharing plans, and the patients themselves. One provider facility 110a is illustrated in FIG. 1 with the provider including a patient intake/accounting system server 105a accessible via a network 115a. The patient intake/accounting system server 105a may be configured with a patient intake/accounting system module 120a to manage the intake of patients for appointments and to generate invoices for payors for services and products rendered through the provider 110a. The network 115a communicatively couples the patient intake/accounting system server 105a to other devices, terminals, and systems in the provider's facility 110a. The network 115a may comprise one or more local or wireless networks to communicate with patient intake/accounting system server 105a when the patient intake/accounting system server 105a is located in or proximate to the health care service provider facility 110a. When the patient intake/accounting system server 105a is in a remote location from the health care facility, such as part of a cloud computing system or at a central computing center, then the network 115a may include one or more wide area or global networks, such as the Internet.

While a patient 102 may seek health care services and/or products from a traditional medical or health care facility, the patient 102 may also seek to use other services that are related to the patient's health care, such as transport services, fitness center memberships, and the like, which are represented as non-traditional health care entities by the bus icon 170. Services and/or products from traditional health care facilities or entities or non-traditional health care entities may be covered as a benefit under a patient's health care coverage plan or policy. A patient may approach a traditional health care facility 110a and/or a non-traditional health care entity 170 to obtain a service, procedure, product, etc. therefrom, but it may be unclear whether the patient's health care plan covers the requested service, etc. as a benefit.

According to some embodiments of the inventive concept, a benefit discovery server 130 may include a benefit discovery module 135 that is configured to provide a coverage eligibility and benefits domain specific model based on rules. These rules may be generated from information from one or more sources, such as payor documentation and/or health care coverage eligibility responses from a payor 160a, 160b. The rules may be stored in a benefit rule repository 140 and may be adjudicated to determine whether the patient 102 is entitled to a particular benefit. The benefit discovery module 135 may also be configured to provide a benefit query recommendation service to assist requestors making benefits eligibility inquiries to generate better and more appropriate eligibility requests based on rules. These rules may be generated from payor documentation and/or health care coverage eligibility responses from a payor 160a, 160b, including, but not limited to, eligibility confirmation responses, prior-authorization for care responses, error responses, and/or default responses. The rules may be stored in an eligibility request rule repository 145 and may be used to evaluate queries to determine whether they can be revised to achieve a more accurate and relevant response from a payor in response to a benefits query or health care coverage eligibility request for a patient. The benefit discovery server 130 may also be configured to identify conditions that may affect a patient's eligibility for a benefit, such as a requirement that the benefit be a medical necessity, e.g., a medical transport benefit is available for end stage renal disease, but only if deemed medically necessary, and/or a requirement that the patient obtain pre-authorization from the payor before exercising the benefit.

A network 150 couples the patient intake/accounting system server 105a and non-traditional health care entities to the benefit discovery server 130. The network 150 may be a global network, such as the Internet or other publicly accessible network. Various elements of the network 150 may be interconnected by a wide area network, a local area network, an Intranet, and/or other private network, which may not be accessible by the general public. Thus, the communication network 150 may represent a combination of public and private networks or a virtual private network (VPN). The network 150 may be a wireless network, a wireline network, or may be a combination of both wireless and wireline networks.

The benefit discovery service and/or the benefit query recommendation service provided through the benefit discovery server 130, the benefit rule repository 140, and the eligibility request rule repository 145 may, in some embodiments, be embodied as a cloud service. For example, health care service providers and/or payors may access the benefit discovery system and/or the benefit query recommendation service as a Web service. In some embodiments, the benefit discovery service may be implemented as a Representational State Transfer Web Service (RESTful Web service).

Although FIG. 1 illustrates an example communication network including a benefit discovery system, it will be understood that embodiments of the inventive subject matter are not limited to such configurations, but are intended to encompass any configuration capable of carrying out the operations described herein.

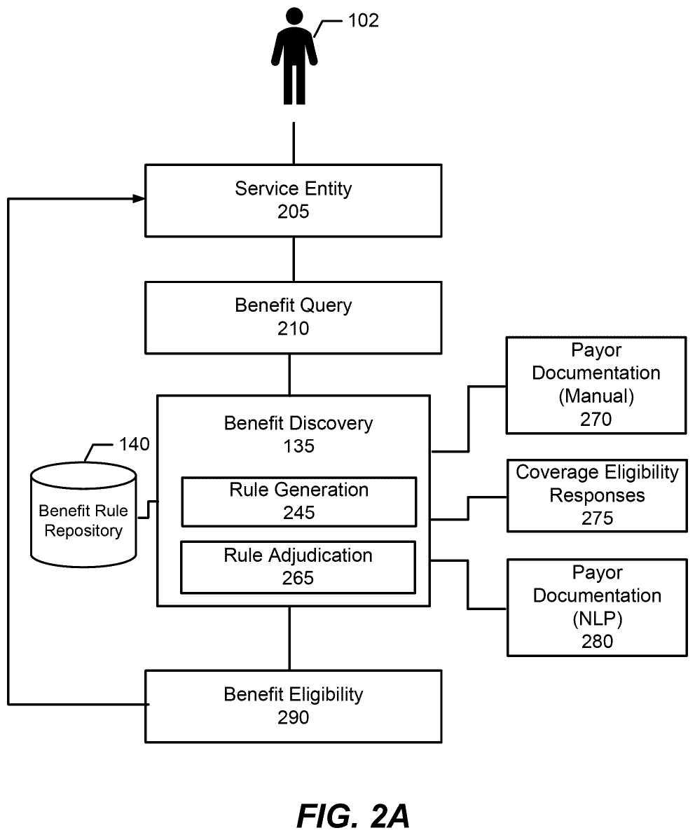

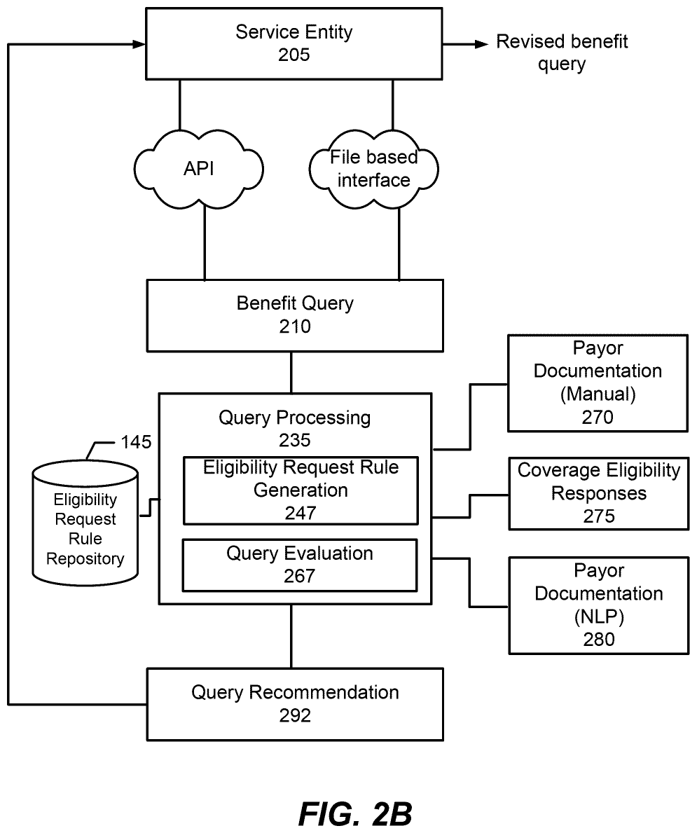

FIG. 2A is a block diagram that illustrates the benefit discovery system in which unstructured information is collected and transformed into structured rules that can be maintained and updated to adjudicate whether a patient is entitled to a benefit under a health care coverage plan in accordance with some embodiments of the inventive concept. Referring to FIG. 2A, a patient 102 may engage a service entity 205, such as a traditional health care service provider 110a and/or a non-traditional health care entity 170 seeking to provide a service, procedure, product, or the like that may be covered as a benefit under the patient's health care coverage plan or policy. For example, a transportation company may wish to determine whether a patient is entitled to a transport benefit under the patient's health care coverage plan. The service entity 205 may use an Application Programming Interface (API) provided by the benefits discovery server to submit a benefit query to the benefit discovery server 130. The benefit query 210 may be passed to the benefit discovery module 135, which may include a rule generation module 245 and a rule adjudication module 265. The rule generation module 245 may be configured to obtain unstructured information from one or more sources and transform this unstructured information into a set of deterministic rules that may be stored in the benefit rule repository 140 for the rule adjudication module 265 to use in adjudicating benefit queries 210. The rule generation module 245 may obtain the unstructured information from one or more benefit information sources including, for example, payor documentation 270, which may be manually reviewed and provided to the rule generation module 245 and/or electronic payor documentation 280, which may be processed using Natural Language Processing (NLP). The payor documentation may comprise a variety of different types or documents that are accessible via a variety of different media types. As an example, the Center for Medicare/Medicaid Services provides a companion guide that articulates business rules associated with coverage information. These rules contain lists of non-computable coverage details for various health care plans for which patients may be eligible. According to some embodiments, the payor documentation may include, but is not limited to, a website or paper document including payor benefit information, an electronic or paper policy document including payor benefit information, and/or an electronic or paper business procedures document including payor benefit information. Paper versions of the documentation may be processed manually or electronically scanned to facilitate NLP processing. The rule generation module 245 may further monitor coverage eligibility responses or transactions from a payor to derive benefits information therefrom. The knowledge contained in a coverage eligibility response may be non-computable; however, the rule generation module 245 may be configured to transform this knowledge from the coverage eligibility responses into a computable representation that may be embodied as a rule that can be used to update the benefit rule repository 140. The rule adjudication module 265 may be configured to adjudicate one or more rules, which are based on a health care coverage policy provided by the payor for the patient 102, contained in the benefit rule repository 140. The benefit eligibility determination 290 may be provided to the service entity 205 including any conditions that may be associated with the benefit, including, but not limited to, a medical necessity condition and/or a payor preauthorization condition as described above.

FIG. 2B is a block diagram that illustrates a benefit query recommendation system in accordance with some embodiments of the inventive concept. The benefit query recommendation system embodiments may be used independently as a service to entities that submit benefit queries or health care coverage eligibility requests to payors and/or may be used in conjunction with the benefits discovery system embodiments of FIG. 2A. A service entity 205, such as a traditional health care service provider 110a and/or a non-traditional health care entity 170 seeking to provide a service, procedure, product, or the like that may be covered as a benefit under the patient's health care coverage plan or policy, may submit a benefit query through various types of interfaces. In the example shown in FIG. 2B, a service entity 205 may submit a benefit query via an API and/or in other embodiments via a file based interface, which may be useful for submitting queries in batches. The benefit query 210 may be passed to the benefit discovery module 135, which may include a query processing module 235. The query processing module 235 may include an eligibility request rule generation module 247 and a query evaluation module 267. The eligibility request rule generation module 247 may be configured to obtain unstructured information from one or more sources and transform this unstructured information into a set of deterministic rules that may be stored in the eligibility request rule repository 145 for the query evaluation module 267 to use in evaluating and suggesting revisions to health care benefit queries. The eligibility request rule generation module 247 may obtain the unstructured information from one or more benefit information sources including, for example, payor documentation 270, which may be manually reviewed and provided to the eligibility request rule generation module 245 and/or electronic payor documentation 280, which may be processed using Natural Language Processing (NLP). The payor documentation may comprise a variety of different types or documents that are accessible via a variety of different media types as described above with respect to FIG. 2A. The eligibility request rule generation module 247 may further monitor coverage eligibility responses or transactions from a payor to derive benefits information therefrom. The knowledge contained in a coverage eligibility response may be non-computable; however, the rule generation module 247 may be configured to transform this knowledge from the coverage eligibility responses into a computable representation that may be embodied as a rule that can be used to update the eligibility request rule repository 140. These payor responses may include, but are not limited to, eligibility confirmation responses, prior-authorization for care responses, error responses, and/or default responses. The query evaluation module 267 may be configured to evaluate a health care benefit query or health care coverage eligibility request submitted by a requestor based on the rules contained in the eligibility request rule repository 145 and to generate a recommendation that contains any suggested revisions that may improve the original query or eligibility request to achieve a more accurate and relevant response from the payor. The query recommendation 292 may be provided to the service entity 205 who may then use the recommendation to generate a revised benefit query.

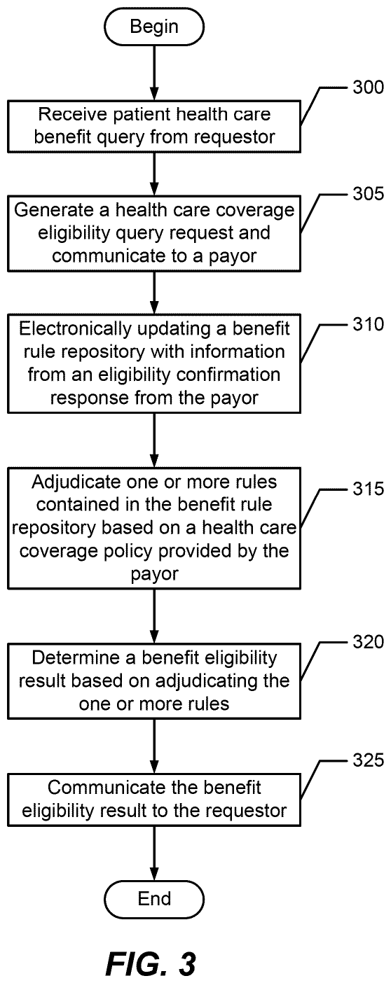

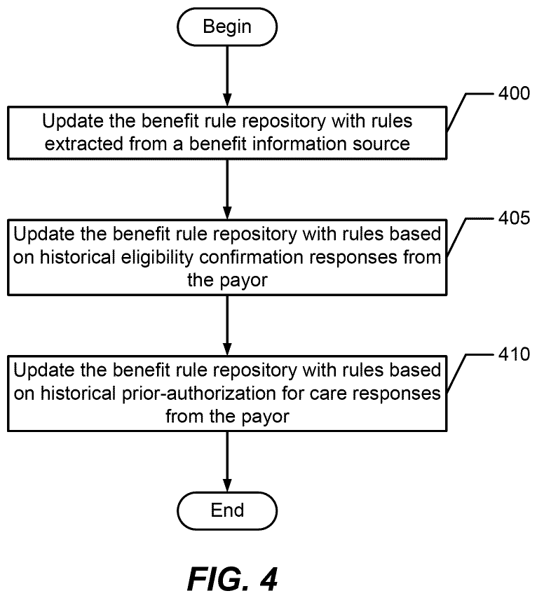

FIGS. 3 and 4 are flowcharts that illustrate operations for discovering benefits under a health care coverage plan in accordance with further embodiments of the inventive concept. Referring to FIG. 3, operations begin at block 300 where a patient health care benefit query is received from a requestor, such as a traditional provider 110a and/or a non-traditional health care entity 170, e.g., a transport service. A heath care coverage eligibility query request may be sent to a payor at block 305. The benefit rule repository 140 may be updated with information from an eligibility confirmation response from the payor at block 310. At block 315, one or more rules contained in the benefit rule repository 140 may be adjudicated, which are based on a health care coverage policy provided by the payor for the patient. A benefit eligibility result may be determined at block 320 and the benefit eligibility result may be communicated to the requestor at block 325. Referring to FIG. 4, at block 400 the benefit rule repository 140 may be updated based on unstructured information obtained from a variety of sources and converted into structured information embodied as rules that may be computed or adjudicated to determine benefit eligibility for a patient. In some embodiments at block 405, the benefit rule repository 140 may be updated with rules that are generated based on historical eligibility confirmation responses from a payor. In further embodiments at block 410, the benefit rule repository may be updated with rules based on historical pre-authorization for care responses from the payor.

The benefit discovery system, according to some embodiments of the inventive concept, may provide a real-time service in which a requestor may obtain an indication whether a patient is entitled to a benefit potentially subject to one or more conditions. Accordingly, the operations of blocks 300 through 325 of FIG. 3 may be performed in real-time, which, as used herein, means without the insertion of any intentional artificial delays between operations.

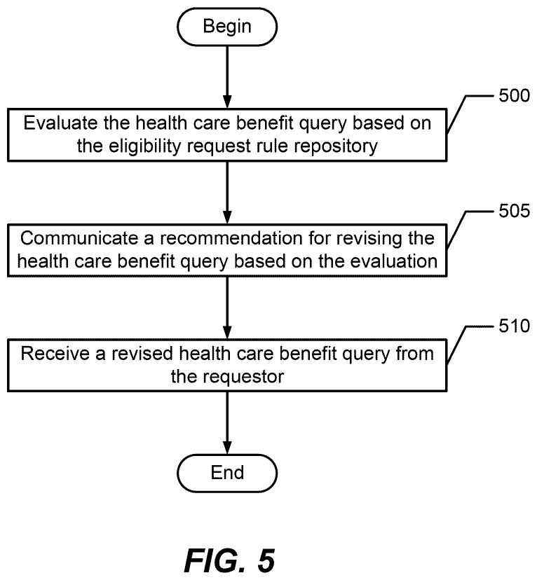

FIG. 5 is a flowchart that illustrates operations for recommending revisions to a benefit query in accordance with some embodiments of the inventive concept. Operations begin at block 500 where the health care benefit query submitted by a requestor is evaluated based on the rules contained in the eligibility request rule repository 145. As described above, the process of submitting benefits queries and/or health care coverage eligibility requests to payors and interpreting the responses is inexact due to the use of the X12 specification. While the queries and responses are defined as 270 queries and 271 responses in the X12 specification, payors and requestors may interpret both the queries and responses in their own way as X12 is merely a specification and not a standard. As a result, in addition to simply a confirmation or denial of the existence of coverage, a payor may also respond with an error message or even a default message that a requestor may interpret as an applicable response to the query, but may instead be inapplicable to the query as originally presented. This may be illustrated by way of example in the context of a customer placing an order at a pizza restaurant that only serves pepperoni and cheese pizzas. A customer places an order for a sausage pizza and the restaurant may acknowledge the order as being accepted and deliver a cheese pizza as a default. In this example, the most useful response would have been an error response to notify the customer than an order was placed for a pizza that the restaurant does not provide. The default response of delivering a cheese pizza misleads the customer into thinking a sausage pizza is being delivered and the customer only realizes the mistake later. Requestors may also get error responses from the payor because the requestor is not aware of how a particular payor interprets certain eligibility request codes and/or may get false no-coverage and false confirmation of coverage responses from a payor because the requestor submitted an eligibility request query that was incomplete or inaccurate in some way. As another example, which pertains to the health care field, a requestor may want to determine if a customer has a benefit for a cabulance service, but submits an eligibility request query to determine if the customer has coverage for a non-emergency transport benefit. Unbeknownst to the requestor, the payor uses two different codes for a cabulance benefit and a non-emergency transport benefit, respectively. Thus, while the customer may indeed have coverage for a cabulance transport, because the requestor submitted a non-emergency transport eligibility query, the payor may provide a no-coverage response. The set of deterministic rules generated and stored in the eligibility request rule repository 145 may be derived from the unstructured information including both payor responses and payor documentation as described above. The rules in both the eligibility request rule repository 145 and the benefit rule repository 140 may, therefore, be payor specific as each payor may have its own set of rules and interpretations for the various 270 benefit eligibility queries and 271 benefit eligibility responses.

At block 505, a recommendation for revising the health care benefit query based on the evaluation of the query in light of the eligibility request rules is communicated to the requestor. Recommendations may be illustrated by way of example. One type of recommendation may be that a particular payor, such as the Center for Medicare and Medicaid Services, does not recognize a particular service type code. Another type of recommendation may be service type code X was used explicitly and this payor returns service type code Y by default. Yet another type of recommendation may be that removing a particular service type code may reduce the number of error responses received from the payor within some established timeframe. These examples are not exhaustive, but are illustrative of the types of recommendations that may be provided for improving benefits or health care coverage eligibility queries or requests. The requestor may then generate a revised query, which may then be resubmitted at block 510.

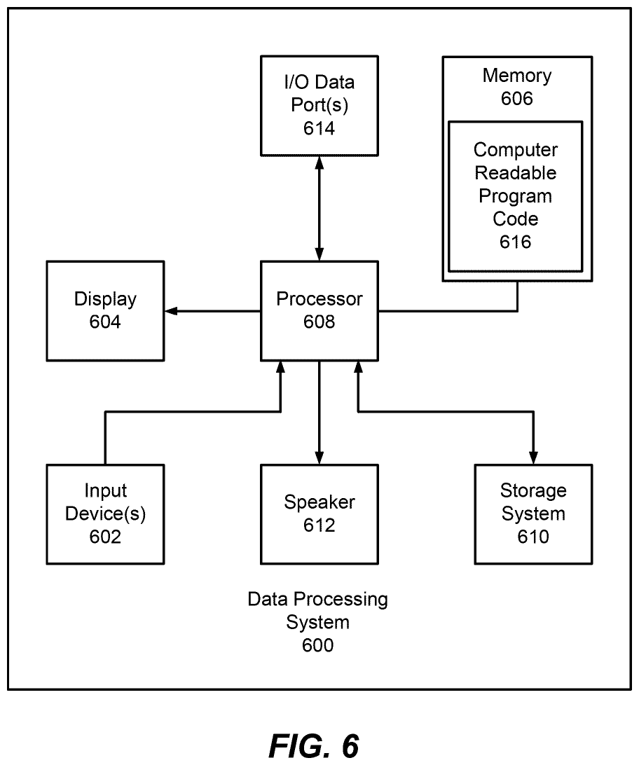

Referring now to FIG. 6, a data processing system 600 that may be used to implement the benefit discovery server 130 of FIG. 1, in accordance with some embodiments of the inventive concept, comprises input device(s) 602, such as a keyboard or keypad, a display 604, and a memory 606 that communicate with a processor 608. The data processing system 500 may further include a storage system 610, a speaker 612, and an input/output (I/O) data port(s) 614 that also communicate with the processor 608. The processor 608 may be, for example, a commercially available or custom microprocessor. The storage system 610 may include removable and/or fixed media, such as floppy disks, ZIP drives, hard disks, or the like, as well as virtual storage, such as a RAMDISK. The I/O data port(s) 614 may be used to transfer information between the data processing system 600 and another computer system or a network (e.g., the Internet). These components may be conventional components, such as those used in many conventional computing devices, and their functionality, with respect to conventional operations, is generally known to those skilled in the art. The memory 506 may be configured with computer readable program code 516 to facilitate discovering patient benefits under a health care coverage plan according to some embodiments of the inventive concept.

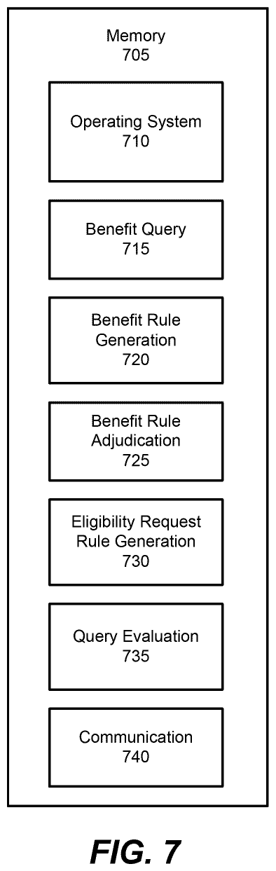

FIG. 7 illustrates a memory 705 that may be used in embodiments of data processing systems, such as the benefit discovery server 130 of FIG. 1 and the data processing system of FIG. 6, respectively, to facilitate discovering patient benefits under a health care coverage plan. The memory 705 is representative of the one or more memory devices containing the software and data used for facilitating operations of the benefit discovery server 130 and the benefit discovery module 135 as described herein. The memory 705 may include, but is not limited to, the following types of devices: cache, ROM, PROM, EPROM, EEPROM, flash, SRAM, and DRAM. As shown in FIG. 7, the memory 705 may contain five or more categories of software and/or data: an operating system 710, a benefit query module 715, a benefit rule generation module 720, a benefit rule adjudication module 725, and a communication module 735. In particular, the operating system 710 may manage the data processing system's software and/or hardware resources and may coordinate execution of programs by the processor. The benefit query module 715 may be configured to perform one or more of the operations described above with respect to the benefit discovery server 130 and benefit discovery module 135, the benefit query block 210, and FIGS. 2A and 2B. The benefit rule generation module 720 may be configured to perform one or more of the operations described above with respect to the benefit discovery server 130 and benefit discovery module 135, the rule generation block 245, FIG. 2A, FIG. 3, and FIG. 4. The benefit rule adjudication module 725 may be configured to perform one or more of the operations described above with respect to the benefit discovery server 130 and benefit discovery module 135, the rule adjudication block 245, FIG. 2A, and FIG. 3. The eligibility request rule generation module 730 may be configured to perform one or more of the operations described above with respect to the benefit discovery server 130 and query processing module 235, the eligibility request rule generation block 247, FIG. 2B, and FIG. 5. The query evaluation module 735 may be configured to perform one or more of the operations described above with respect to the benefit discovery server 130 and query processing module 235, the query evaluation block 267, FIG. 2B, and FIG. 5. The communication module 740 may be configured to facilitate communication between the benefit discovery server 130 and a service entity, such as a traditional provider 110a and/or a non-traditional health care entity 170 and between the benefit discovery server 130 and payors 160a, 160b.

Although FIG. 7 illustrates hardware/software architectures that may be used in data processing systems, such as the benefit discovery server 130 of FIG. 1 and the data processing system of FIG. 6, respectively, in accordance with some embodiments of the inventive concept, it will be understood that embodiments of the present invention are not limited to such a configuration but is intended to encompass any configuration capable of carrying out operations described herein.

Computer program code for carrying out operations of data processing systems discussed above with respect to FIGS. 1-7 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the benefit discovery server 130 and the data processing system 600 of FIG. 6 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.” The functionality provided by the benefit discovery server 130 may be embodied as a single server or embodied as separate servers in accordance with different embodiments of the inventive concept.

The data processing apparatus described herein with respect to FIGS. 1-6 may be used to facilitate discovery of patient benefits under a health care coverage plan according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 605 when coupled to a processor includes computer readable program code that, when executed by the processor, causes the processor to perform operations including one or more of the operations described herein with respect to FIGS. 1-5

Some embodiments of the inventive concept may provide a benefit discovery system that may be configured to collect unstructured information from multiple disparate sources, such as coverage eligibility responses and various payor documentation sources, and creating a structured, domain specific model based on rules that can be adjudicated. The unstructured information may be obtained from a variety of sources including, but not limited to, payor eligibility confirmation responses, NLP analysis of electronic payor documentation, and/or manual analysis or paper or electronic payor documentation. The benefit discovery system may allow benefit eligibility queries to be processed more efficiently with improved consistency.

### Further Definitions and Embodiments

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving a patient health care benefit query from a requestor;
generating a health care coverage eligibility request for the patient;
communicating the health care coverage eligibility request to a payor;
electronically updating a benefit rule repository with information from an eligibility confirmation response from the payor;
adjudicating one or more rules, which are based on a health care coverage policy provided by the payor for the patient, contained in the benefit rule repository;
determining a benefit eligibility result for the patient based on adjudicating the one or more rules; and
communicating the benefit eligibility result to the requestor.

2. The method of claim 1, further comprising:
processing a benefit information source associated with the payor using Natural Language Processing (NLP) to extract rules therefrom; and
updating the benefit rule repository with the rules extracted from the benefit information source.

3. The method of claim 2, wherein the benefit information source comprises a website including payor benefit information, an electronic policy document including payor benefit information, and/or an electronic business procedures document including payor benefit information.

4. The method of claim 1, further comprising:
manually processing a benefit information source associated with the payor to extract rules therefrom; and
updating the benefit rule repository with the rules extracted from the benefit information source.

5. The method of claim 4, wherein the benefit information source comprises a website or paper document including payor benefit information, an electronic or paper policy document including payor benefit information, and/or an electronic or paper business procedures document including payor benefit information.

6. The method of claim 1, further comprising:
updating the benefit rule repository with rules based on historical eligibility confirmation responses from the payor for historical patients; and
updating the benefit rule repository with rules based on historical prior-authorization for care responses from the payor for historical patients.

7. The method of claim 1, wherein the operations of receiving the patient health care benefit query, generating the health care coverage eligibility request, communicating the health care coverage eligibility request, electronically updating the benefit rule repository, adjudicating the one or more rules, determining the benefit eligibility result, and communicating the benefit eligibility result are performed in real-time.

8. The method of claim 1, wherein the benefit eligibility result comprises one or more conditions associated therewith.

9. The method of claim 8, wherein the one or more conditions are based on clinical information associated with the patient, whether the benefit qualifies as a medical necessity, and/or whether the payor requires pre-authorization for the benefit.

10. The method of claim 1, wherein generating the health care coverage eligibility request for the patient comprises:
processing a benefit information source associated with the payor using Natural Language Processing (NLP) to extract rules therefrom or manually processing the benefit information source associated with the payor to extract the rules therefrom; and
updating an eligibility request rule repository with the rules extracted from the benefit information source.

11. The method of claim 10, wherein generating the health care coverage eligibility request for the patient further comprises:
updating the eligibility request rule repository with rules based on historical responses from the payor for historical patients;
wherein the historical responses from the payor comprise historical eligibility confirmation responses from the payor for historical patients, historical prior-authorization for care responses from the payor for historical patients, eligibility query error responses from the payor for historical patients, and eligibility default responses from the payor for historical patients.

12. The method of claim 11, wherein generating the health care coverage eligibility request for the patient further comprises:
evaluating the health care benefit query based on the eligibility request rule repository to generate an evaluation of the health care benefit query;
communicating a recommendation for revising the health care benefit query based on the evaluation of the health care benefit query; and
receiving a revised health care benefit query from the requestor;
wherein communicating the health care coverage eligibility request to the payor comprises communicating the health care coverage eligibility request to the payor based on the revised health care benefit query from the requestor.

13. A system, comprising:
a processor; and
a memory coupled to the processor and comprising computer readable program code embodied in the memory that is executable by the processor to perform operations comprising:
receiving a patient health care benefit query from a requestor;
generating a health care coverage eligibility request for the patient;
communicating the health care coverage eligibility request to a payor;
electronically updating a benefit rule repository with information from an eligibility confirmation response from the payor;
adjudicating one or more rules, which are based on a health care coverage policy provided by the payor for the patient, contained in the benefit rule repository;
determining a benefit eligibility result for the patient based on adjudicating the one or more rules; and
communicating the benefit eligibility result to the requestor.

14. The system of claim 13, wherein the operations further comprise:
processing a benefit information source associated with the payor using Natural Language Processing (NLP) to extract rules therefrom; and
updating the benefit rule repository with the rules extracted from the benefit information source.

15. The system of claim 14, wherein the benefit information source comprises a website including payor benefit information, an electronic policy document including payor benefit information, and/or an electronic business procedures document including payor benefit information.

16. The system of claim 13, wherein the operations further comprise:
manually processing a benefit information source associated with the payor to extract rules therefrom; and
updating the benefit rule repository with the rules extracted from the benefit information source.

17. The system of claim 16, wherein the benefit information source comprises a website or paper document including payor benefit information, an electronic or paper policy document including payor benefit information, and/or an electronic or paper business procedures document including payor benefit information.

18. The system of claim 13, wherein the operations further comprise:
updating the benefit rule repository with rules based on historical eligibility confirmation responses from the payor for historical patients; and
updating the benefit rule repository with rules based on historical prior-authorization for care responses from the payor for historical patients.

19. A computer program product, comprising:
a non-transitory computer readable storage medium comprising computer readable program code embodied in the medium that is executable by a processor to perform operations comprising:
receiving a patient health care benefit query from a requestor;
generating a health care coverage eligibility request for the patient;
communicating the health care coverage eligibility request to a payor;
electronically updating a benefit rule repository with information from an eligibility confirmation response from the payor;
adjudicating one or more rules, which are based on a health care coverage policy provided by the payor for the patient, contained in the benefit rule repository;
determining a benefit eligibility result for the patient based on adjudicating the one or more rules; and
communicating the benefit eligibility result to the requestor.

20. The computer program product of claim 19, wherein the operations further comprise:
processing a benefit information source associated with the payor using Natural Language Processing (NLP) to extract rules therefrom; and
updating the benefit rule repository with the rules extracted from the benefit information source.
